(19) United States
(12) Patent Application Publication
(10) Pub. No.: US 2016/0061813 A1
(43) Pub. Date: Mar. 3, 2016

(54) PORTABLE DEVICE FOR ANALYSING pH OR ANOTHER VARIABLE BY ELECTROCHEMICAL MEASUREMENT

(71) Applicant: Universitat de les Illes Balears, Palma de Mallorca (ES)

(72) Inventors: Isabel GOMILA MUÑOZ, Palma de Mallorca (ES); Feliciano Grases Freixedas, Palma de Mallorca (ES); Antonia Costa-Bauza, Palma de Mallorca (ES); Rafael M. Prieto Almirall, Palma de Mallorca (ES); Eva Martin Becerra, Palma de Mallorca (ES); Ruben Henriquez Pelaez, Palma de Mallorca (ES); Bernat Isern Amengual, Palma de Mallorca (ES); Joan Perello Bestard, Palma de Mallorca (ES)

(21) Appl. No.: 14/843,656

(22) Filed: Sep. 2, 2015

Related U.S. Application Data
(63) Continuation of application No. 13/515,719, filed on Aug. 9, 2012.

(30) Foreign Application Priority Data
Dec. 14, 2009 (ES) U200930750

(51) Int. Cl. G01N 33/493; G01N 27/30

(52) U.S. Cl. CPC G01N 33/493; G01N 27/302

(57) ABSTRACT

Portable device (1) for analysing pH or another variable by electrochemical measurement comprising a sensor (2), a microprocessor (3) and display means (4) for the results of the analysis. The portable device makes it possible to carry out pre-diagnoses quickly and easily without the need for specialist staff.

This invention is a portable device for analysing pH or another variable by electrochemical measurement, which makes it possible to carry out pre-diagnoses quickly and easily without the need for specialist staff.

### BACKGROUND OF THE INVENTION

Renal lithiasis is a pathology caused by the crystalisation of different substances in the urinary tract. More specifically, the crystalisation of uric acid always occurs in urine with a pH lower than 5.5. There are various factors that contribute to the formation of kidney stones, as well as hyperuricosuria.

It must be taken into account that the pKa of uric acid is approximately 5.5. This is the pH at which the ionic and non-ionic species of uric acid are balanced. Therefore, urine with a pH lower than 5.5 presents a higher proportion of undissociated (insoluble) forms that facilitate the formation of uric acid crystals and kidney stones.

On the other hand, the crystalisation of calcium phosphate in the form of hydroxyapatite [Ca₁₀(PO₄)₆(OH)₂], always occurs at a urinary pH higher than 6. There are other factors that contribute to the formation of hydroxyapatite stones, such as the presence or absence of crystalisation inhibitors or supersaturation, as a thermodynamic factor.

Finally, the crystalisation of calcium oxalate is caused by heterogeneous nucleation where, at a very high percentage, uric acid crystals or hydroxyapatite crystals can act as heterogeneous nuclei in the formation of calcium oxalate crystals.

One of the problems to be solved is the lack of portable devices that can easily and effectively measure the risk of kidney stones forming (consequence of urinary pH or other altered factors) in the urine of lithiasic patients or in healthy people who wish to prevent calcium oxalate, uric acid and hydroxyapatite lithiasis.

Although this check could be carried out using pH indicator paper or a similar system, we must point out that the precision of said systems is not enough to distinguish 0.1 pH units and therefore it would be difficult to obtain a diagnostic value regarding the risk of developing lithiasis.

In addition, the majority of pH metres with a digital or analogue indicator are difficult for patients to use, interpret and maintain and involve a high cost for individual use.

### DESCRIPTION OF THE INVENTION

To achieve the foregoing objects, this invention is a portable device for analysing pH or another variable by electrochemical measurement in accordance with claim 1, the use of a portable analysis device in accordance with claim 15 and a method for measuring pH or another variable by electrochemical measurement in accordance with claim 19. In the dependent claims, preferred embodiments of this invention are defined.

In a preferred embodiment the portable pH analysis device comprises a pH sensor, a microprocessor and display means for the results of the analysis, and is characterised for having three light indicators, the microprocessor being configured to activate:
the first of these indicators when the pH is lower than an initial pH threshold,
the second of these indicators when the pH is higher than a second pH threshold and,
the third of these indicators when the pH is between the abovementioned first and second thresholds.

Thus, this device will be useful to lithiasic patients, in that they can determine for themselves and with precision the effects of a diet and even food, drinks and dietary supplements, medicines or physical activity on the risk of kidney stone formation. More specifically, for patients who present a clear risk of lithiasis from uric acid or hydroxyapatite, as well as for those who are at risk of oxalocalcium lithiasis. We must point out that preventing the crystalisation of uric acid and hydroxyapatite would also indirectly prevent oxalocalcium lithiasis in a very high percentage of cases.

Preferably, the device in accordance with the invention is configured in the form of an adaptable lid for a urine specimen collection cup, the pH sensor being arranged to enter into contact with a urine sample inside the cup, once the device is affixed to the cup, thus allowing the device to be used under hygienic conditions and providing an effective reading, where possible placing the electrode in a suitable position with regard to the sample.

Ideally, the first pH threshold is 5.5 and the second threshold is 6.

Advantageously, the pH sensor comprises a pH electrode and a reference electrode for measuring the proton electrochemical potential in the urine.

A further benefit is that the device includes means for measuring the temperature in the pH and reference electrodes.

Finally, on the device of the invention, the first indicator is red, the second is blue and the third is green.

The device can be fitted with additional sensors for measuring a variable by electrochemical measurement such as calcium or redox potential.

In a second aspect of the invention, the use of a device is defined in accordance with the first aspect of the invention for measuring pH or another variable by electrochemical measurement in a biological fluid sample. The biological fluid is preferably urine, although it may be another fluid, for example blood, saliva or sputum.

In a third aspect of the invention a method of measuring pH or another variable by electrochemical measurement of a sample is defined, which involves measuring pH or another variable by electrochemical measurement of the sample and providing an indication of the results of the measurement, depending on it being within at least one range of values.

### BRIEF DESCRIPTION OF THE DRAWINGS

In order to achieve a better understanding of what has been presented, some drawings have been attached in which a practical case of use is represented schematically and only as a non-limitative example.

FIG. 1 is a cross section of the preferred embodiment of the invention in which it is configured in the form of a lid affixed to a specimen cup.

FIG. 2 is a block diagram showing the essential elements of the device of the invention.

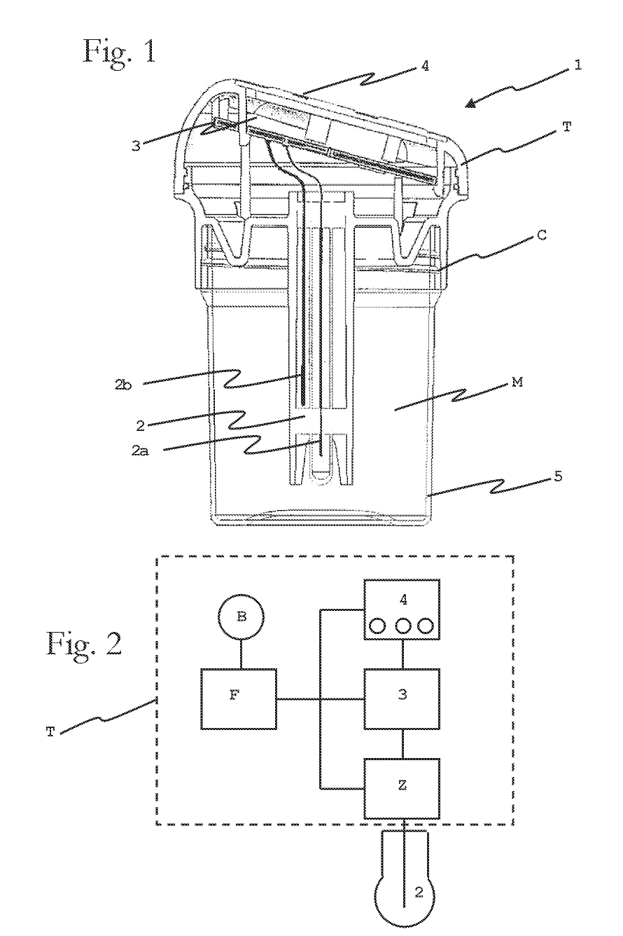

### DESCRIPTION OF A PREFERRED EMBODIMENT

As illustrated in the diagrams, in a particular embodiment the invention refers to a portable device 1 for analysing pH in urine M with a pH sensor 2, a microprocessor 3 and display means 4 for the results of the analysis.

More specifically, the invention is characterised for having three light indicators 4a, 4b and 4c, the microprocessor being configured to activate:
the first of these indicators 4a when the pH is lower than an initial pH threshold,
the second of these indicators 4b when the pH is higher than a second pH threshold and,
the third of these indicators 4c when the pH is between the abovementioned first and second thresholds.

Thus, any user can carry out a pre-diagnosis without the need to seek medical advice. Likewise, this could also be a pre-diagnosis tool for medical staff.

In accordance with the preferred embodiment of the invention, the device is configured in the form of a lid T which can be adapted to a urine M specimen cup 5.

The pH sensor 2 is arranged to enter into contact with a urine sample M contained in the cup 5, when the device 1 is attached to the cup 5, in such a way that adequate hygiene conditions are ensured and the correct placement of the electrodes is guaranteed with regards to the urine sample collected in the cup 5.

As has already been specified, the first threshold is 5.5, meaning that a lower reading indicates the capacity of nucleation and formation of uric acid crystals, which at the same time can become growth nuclei for calcium oxalate crystals. The second threshold is 6, meaning that the detection of a pH higher than this second threshold indicates the capacity of nucleation and growth of hydroxyapatite crystals, which can at the same time act as a nucleant for the calcium oxalate crystals.

The pH sensor 2 comprises a pH electrode 2a and a reference electrode 2b for measuring the proton electrochemical potential in the urine.

Calibration of the equipment is very simple, it only being necessary to replace the measuring cup with a test cup, which must contain a standard solution.

Likewise, in accordance with this preferred embodiment, the device contains means for measuring the temperature in the pH electrodes 2a and reference electrodes 2b.

As can be seen in diagram 2, the reading electrodes 2 protrude from the lid, and housed inside the lid is the battery B, the power source F, the electrode impedance adaptor Z, the microprocessor 3 and the indicator lights 4, connected together as shown in the diagram.

Using the device is extremely simple and consists of the following stages:
1. Separate the cup from the rest of the instrument by unscrewing the lid.
2—The patient deposits the urine in the cup
3—Screw the lid back on and shake lightly.
4—Press a test key.
5—In accordance with this preferred embodiment, the three lights will blink in sequence for a few seconds to indicate that the measurement is being taken. The result has been obtained when one of the lights stays lit.
6—Unscrew the lid, throw away the contents and wash both the cup, as well as the lid, with plenty of water before using again.
7—Screw the two parts together again so that some drops of water stay inside.

1-20. (canceled)

21. A method for self-diagnosis of lithiasis in a subject in need thereof comprising:
(i) contacting a urine sample obtained from said subject with a portable device comprising an electrochemical sensor; and,
(ii) measuring pH and/or ionic calcium in said urine sample using the electrochemical sensor in said portable device, thereby providing a measurement value of pH and/or ionic calcium;
wherein a measurement of pH and/or ionic calcium above or below at least one threshold value is indicative of lithiasis or risk of lithiasis.

22. The method according to claim 21, wherein the electrochemical sensor is mounted on a lid affixable to a specimen collection cup.

23. The method according to claim 21, wherein the electrochemical sensor comprises a pH sensor, a calcium sensor, or a combination thereof.

24. The method according to claim 21, wherein the electrochemical sensor further comprises a redox potential sensor, and the variable indicative of lithiasis or risk of lithiasis further comprises redox potential.

25. The method according to claim 21, wherein the lithiasis is uric acid lithiasis, hydroxyapatite lithiasis, oxalocalcium lithiasis, or a combination thereof.

26. The method according to claim 21, wherein the method comprises one threshold value.

27. The method according to claim 21, wherein the method comprises two threshold values.

28. The method according to claim 27, wherein the two threshold values correspond to pH.

29. The method according to claim 27, wherein one threshold value corresponds to pH and an one threshold value corresponds to ionic calcium.

30. The method according to claim 28, wherein the first threshold value is indicative of nucleation and formation of uric acid crystals.

31. The method according to claim 28, wherein the second threshold value is indicative of capacity of nucleation and growth of hydroxyapatite crystals.

32. The method according to claim 30, wherein the first threshold value is pH 5.5 and a measurement value lower than said threshold value is indicative of lithiasis or risk of lithiasis, and wherein said lithiasis is uric acid lithiasis.

33. The method according to claim 33, wherein the second threshold value is pH 6 and a measurement value higher than said threshold value is indicative of lithiasis or risk of lithiasis, and wherein said lithiasis is hydroxyapatite lithiasis.

34. The method according to claim 21, wherein the subject is a human subject.
